(19) United States
(12) Patent Application Publication
(10) Pub. No.: US 2011/0004203 A1
(43) Pub. Date: Jan. 6, 2011

(54) Laser Device and Method for Decreasing the Size and/or Changing the Shape of Pelvic Tissues

(76) Inventor: Ralph Zipper, Melbourne, FL (US)

Correspondence Address: HAYWORTH, CHANEY & THOMAS, P.A., 202 N. HARBOR CITY BLVD., SUITE 300, MELBOURNE, FL, 32935, US

(21) Appl. No.: 12/687,965

(22) Filed: Jan. 15, 2010

Related U.S. Application Data
(63) Continuation-in-part of application No. 12/496,216, filed on Jul. 1, 2009.

(51) Int. Cl. A61B 18/22

(52) U.S. Cl. 606/15

(57) ABSTRACT

Methods for decreasing the size and/or changing the shape of pelvic tissues. In one embodiment a cannula needle having a laser fiber disposed therein is used to delivery energy to the surrounding pelvic tissue. The cannula needle may be advanced into the pelvic tissue, such as the vagina, with the laser fiber in a retracted position and retained within the distal tip of the cannula needle for protection. Upon reaching the initial treatment zone, the laser fiber may be advanced or motivated into an extended position where the distal end of the laser fiber extends beyond the distal tip of the cannula. Laser energy may then be applied as the device is withdrawn from the patient. The application of such energy may be constant, intermittent, increasing, and/or decreasing as needed. The energy application may also be deactivated if a maximum activation time, maximum temperature, or maximum energy level is reached.

### CROSS REFERENCE TO RELATED APPLICATIONS

This application is a continuation-in-part of Ser. No. 12/496,216, filed with the USPTO on Jul. 1, 2009, which is herein incorporated by reference in its entirety.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISK

Not applicable.

### BACKGROUND OF THE INVENTION

1. Field of the Invention

The present invention generally relates to surgical methods, more specifically, the present invention relates to changing the shape and/or size of tissues and structures within the pelvic region including but not limited to the vagina, labia, prepuce, perineum, and other supportive tissues.

2. Background Art

Many women are unhappy with the size, shape, and/or contour of the vagina or labia. This may be secondary to changes that occur with childbirth, vaginal or pelvic surgery, and/or aging. Sometimes the size, shape, and/or contour abnormality may be congenital. This enlargement and/or unsatisfactory shape or contour may lead to sexual dysfunction which may be anatomic or psychological in nature. Until recently, vaginal reconstruction and vulvar surgery has been reserved for the treatment of neoplasia and prolapse. As women have become more outspoken about their dissatisfaction with their genitalia, surgeons have begun to offer those patients surgical corrections typically utilized for the treatment of neoplasia and prolapse. Although these surgeries may alter the size and shape of the vagina and labia, they may often compromise sexual function or create less than optimal aesthetic results.

Presently utilized surgeries injure tissue, deform anatomy, or remove vital tissue. The sexual dysfunction created by such surgeries may be secondary to stenosis of the vagina, shortening of the vagina, injury to muscles or nerves leading to pain or anesthesia, injury of the Graffenberg Spot, removal of the Graffenberg spot, or poor aesthetic appearance leading to psychological sexual dysfunction.

Injuries to the supporting structures of the vagina and surrounding tissues may also cause urinary incontinence. Present treatments for urinary incontinence do not restore normal anatomic structure. Such treatments either create new support with donor or synthetic tissue or distort anatomy to create a compensatory mechanism for managing the defect.

### BRIEF SUMMARY OF THE INVENTION

In accordance with one embodiment, a method for reshaping tissue, the method comprising the steps of providing a device comprising a cannula needle and a laser fiber coaxially disposed within the cannula needle, wherein the laser fiber may be disposed in a retracted position with a distal end of the laser fiber disposed within a distal tip of the cannula needle and an extended position wherein the distal end of the laser fiber protrudes beyond the distal tip of the cannula needle, advancing the cannula needle into the tissue wherein the laser fiber is in the retracted position, wherein the cannula needle forms a pathway within the tissue during the advancement, motivating the laser fiber into the extended position, activating the laser fiber thereby delivering laser energy to the adjacent tissue, and withdrawing the cannula needle along the pathway providing for delivery of the laser energy along the pathway during the withdrawal.

In accordance with another embodiment of the present invention, a method for reshaping pelvic tissue, the method comprising the steps of providing a device comprising a cannula needle and a laser fiber coaxially disposed within the cannula needle, wherein the laser fiber may be disposed in a retracted position with a distal end of the laser fiber disposed within a distal tip of the cannula needle and an extended position wherein the distal end of the laser fiber protrudes beyond the distal tip of the cannula needle, advancing the cannula needle into the pelvic tissue wherein the laser fiber is in the retracted position, wherein the cannula needle forms a pathway within the pelvic tissue during the advancement, motivating the laser fiber into the extended position, activating the laser fiber thereby delivering laser energy to the pelvic tissue, and withdrawing the cannula needle along the pathway providing for delivery of the laser energy along the pathway during the withdrawal, wherein the laser fiber is biased by a spring into the extended position, wherein during the advancement of the cannula needle the laser fiber is moved back against said spring into said retracted position and during said withdrawal of said cannula needle the spring biases the laser fiber into the extended position.

In accordance with another embodiment of the present invention, a method for reshaping pelvic tissue, the method comprising the steps of providing a device comprising a cannula needle and a laser fiber coaxially disposed within the cannula needle, wherein the laser fiber may be disposed in a retracted position with a distal end of the laser fiber disposed within a distal tip of the cannula needle the an extended position wherein the distal end of the laser fiber protrudes beyond the distal tip of the cannula needle, advancing the cannula needle into the pelvic tissue wherein the laser fiber is in the retracted position, wherein the cannula needle forms a pathway within the pelvic tissue during the advancement, motivating the laser fiber into the extended position, activating the laser fiber thereby delivering laser energy to the pelvic tissue, and withdrawing the cannula needle along the pathway providing for delivery of the laser energy along the pathway during the withdrawal, wherein the laser fiber is manually motivated between the retracted position and the extended position by a slide limiter on the device.

### BRIEF DESCRIPTION OF THE DRAWINGS

FIG. 1A depicts a schematic diagram of one step in a first embodiment of the present invention.

FIG. 1B depicts a schematic diagram of another step in the first embodiment of the present invention.

FIG. 1C depicts a schematic diagram of still another step in the first embodiment of the present invention.

FIG. 1D depicts a schematic diagram of yet another step in the first embodiment of the present invention.

FIG. 2A depicts a schematic diagram of one step in a second embodiment of the present invention.

FIG. 2B depicts a schematic diagram of another step in the second embodiment of the present invention.

FIG. 2C depicts a schematic diagram of still another step in the second embodiment of the present invention.

FIG. 3A depicts a schematic diagram of one step in a third embodiment of the present invention.

FIG. 3B depicts a schematic diagram of another step in the third embodiment of the present invention.

FIG. 3C depicts a schematic diagram of still another step in the third embodiment of the present invention.

FIG. 4A depicts a schematic diagram of a treatment phase of a fourth embodiment of the present invention.

FIG. 4B depicts a schematic diagram of a post-treatment phase of the fourth embodiment of the present invention.

FIG. 5A depicts a side view of an embodiment of a laser energy source of the present invention.

FIG. 5B depicts a magnified side view of the embodiment of the laser energy source of the present invention depicted in FIG. 5A.

FIG. 6A depicts a schematic diagram of a treatment phase of a fifth embodiment of the present invention.

FIG. 6B depicts a schematic diagram of a post-treatment phase of the fifth embodiment of the present invention.

FIG. 7 depicts a side view of another embodiment of a laser energy source of the present invention comprising a laser scalpel.

FIG. 8 depicts a side view of another embodiment of a laser energy source of the present invention.

### DETAILED DESCRIPTION OF THE INVENTION

The scope and breadth of the present inventive disclosure is applicable across a wide variety of procedures, tissues and anatomical structures. Although the following detailed description contains many specifics for the purposes of illustration, anyone of ordinary skill in the art will appreciate that many variations and alterations to the following details are within the scope of the invention. Accordingly, the following preferred embodiments of the invention are set forth without any loss of generality to, and without imposing limitations upon, the claimed invention.

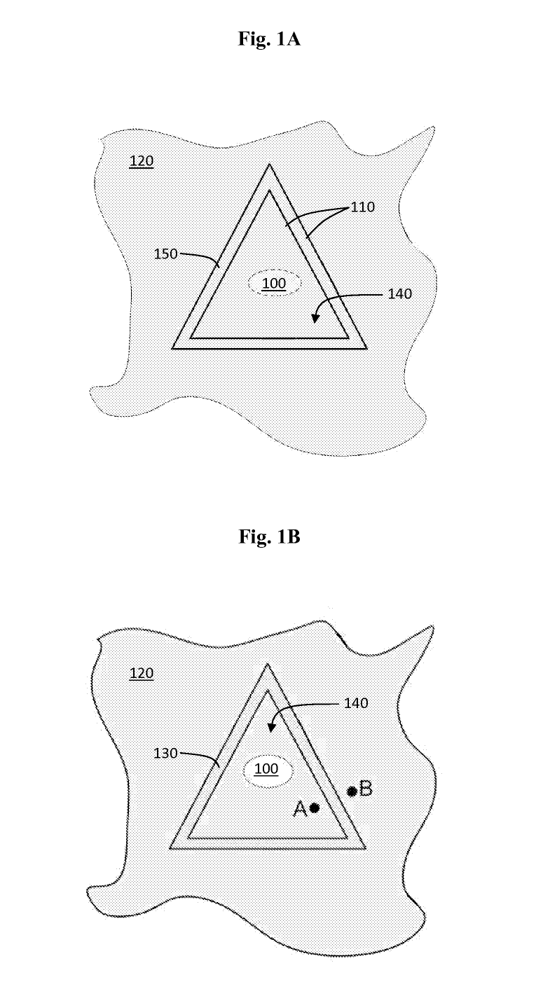

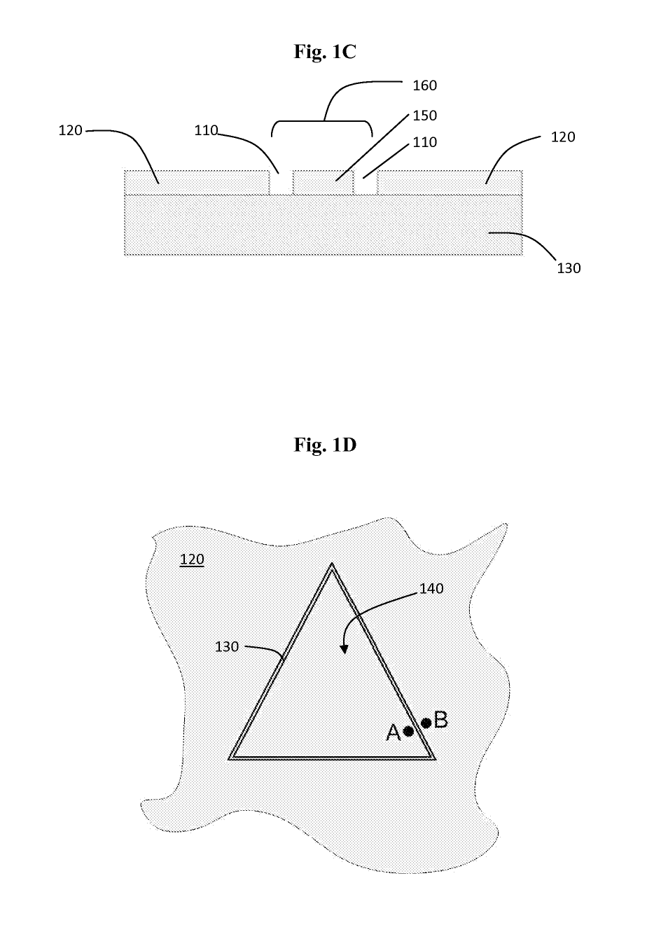

A first embodiment, depicted in FIGS. 1A-1D, may provide for protection of the Graffenberg Spot (G-Spot). In this embodiment, the vaginal mucosa of the G-Spot 100 may be left intact. At least one incision 110 of any known shape, preferably triangular-shaped, may be made around the G-Spot 100, as shown in FIG. 1A. The at least one incision 110 may be carried through the thickness of the vaginal mucosa 120. The at least one incision 110 may spare the endopelvic fascia 130. A preferably triangular-shaped island 140 of mucosa 120 may then be created. A strip 150 of mucosa 120 may be removed from the circumference of the island 140 to expose a channel 160 of endopelvic fascia 130, as shown in FIG. 1B and FIG. 1C. The diameter of this channel 160 will determine the final shape and/or size of the vagina. As shown in FIG. 1D, radio frequency (RF) energy may then be applied to shrink the channel 160 of endopelvic fascia 130 and close the gap between the mucosal 120 edges as shown by the relative movement of point A and point B. The limited penetration of RF energy spares the underlying nerve structure and improves the thickness of peri-island fascia. The mucosal 120 edges may be left “as is”, approximated with sutures or glue, or closed by any other manner known within the art. Although RF is the preferred energy source, any other types of energy known within the art including but not limited to laser, microwave, and monopolar or bipolar electrosurgery may be used.

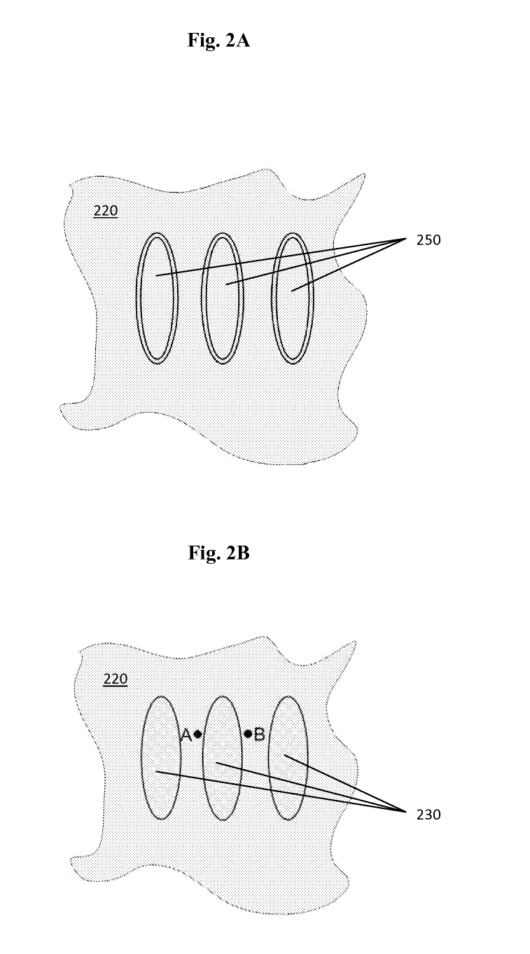

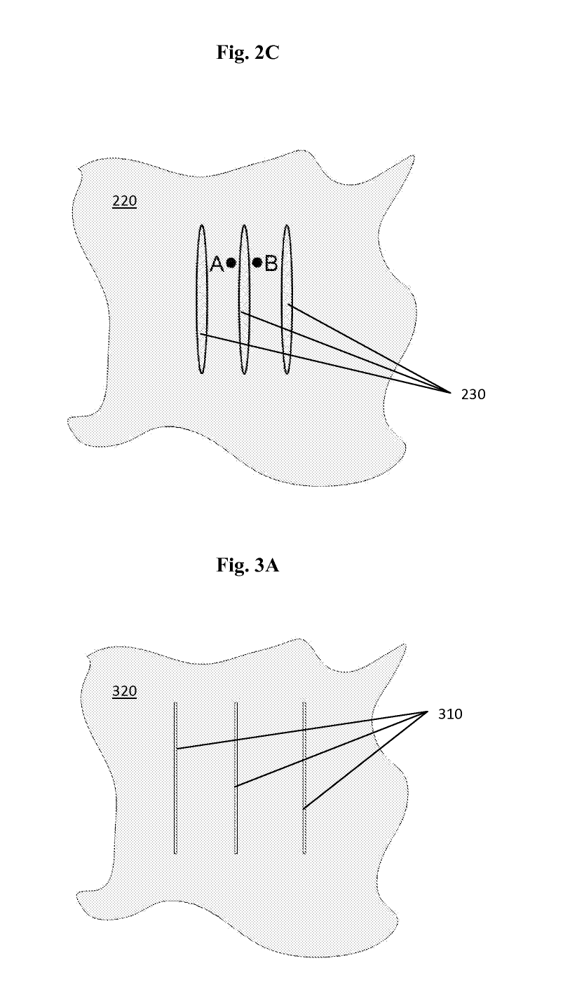

A second embodiment, depicted in FIGS. 2A-2C, may provide for vaginal shaping without removal of fascia. In this embodiment, as shown in FIG. 2A, strips 250 of vaginal mucosa 220 may be removed while sparing the underlying endopelvic fascia 230 and nerve injury (see FIG. 2B). Rather than pulling the mucosal 220 edges together and creating a submucosal deformity, RF energy may be applied to shrink the endopelvic fascia 230 and bring the mucosal 220 edges closer together, as shown by the relative movement of point A and point B in FIGS. 2B and 2C. The limited penetration of RF energy acts to spare the underlying nerve structure and improves the thickness of underlying tissue. The mucosal 220 edges may be left “as is”, approximated with sutures or glue, or closed by any other manner known within the art. Although RF is the preferred energy source, any other types of energy known within the art including but not limited to laser, microwave, and monopolar or bipolar electrosurgery may be used.

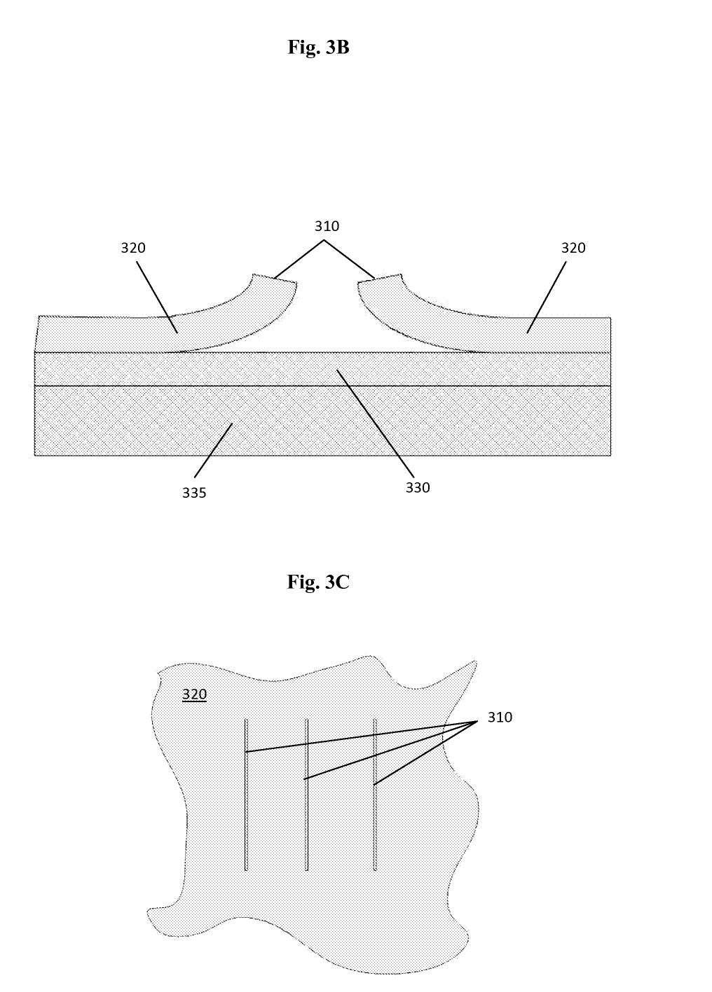

A third embodiment, depicted in FIG. 3A-3C, may provide for vaginal shaping without removal of mucosa. As shown in FIG. 3A, one or more incisions 310 may be made in the mucosa 320. The endopelvic fascia 330 or other submucosal tissue may be left attached to the mucosa 320. As shown in FIG. 3B, RF energy may be applied to the endopelvic fascia 330 or other submucosal tissue exposed between the incision 310 margins. Such an application of energy will cause shrinkage of such endopelvic fascia 330 tissue with proportional contraction of the overlying mucosa 320 and spare the deep nerves and subfascial or subcutaneous tissue 335. Any such endopelvic fascia 330 that is left exposed (as expressly disclosed in all embodiments) may be treated with RF energy. In this manner, the mucosal 320 edges closer together and provide a new contour or shape to the mucosa 320, as shown in FIG. 3C. The mucosal 320 edges may be left “as is”, approximated with sutures or glue, or closed by any other manner known within the art. Although RF is the preferred energy source, any other types of energy known within the art including but not limited to laser, microwave, and monopolar or bipolar electrosurgery may be used.

A fourth embodiment may provide for contouring of the prepuce. As expressly disclosed in the method steps above, an incision may be created around the prepuce and RF energy may thereafter be applied to the underlying fascia. Such an embodiment is similar to that shown in FIGS. 3A-3C and analogous steps may be applied to the prepuce. Although RF is the preferred energy source, any other types of energy known within the art including but not limited to laser, microwave, and monopolar or bipolar electrosurgery may be used.

A fifth embodiment may provide for contouring of the labia minora. As expressly disclosed in the method steps above, an incision may be made in the labia minora. The subcutaneous tissue may not be separated from the epithelium. RF energy may then be applied to the subcutaneous tissue. The shrinkage of the subcutaneous tissue and/or fascia shall contour the labia. Such an embodiment is similar to that shown in FIGS. 3A-3C and analogous steps may be applied to the labia minora. Although RF is the preferred energy source, any other types of energy known within the art including but not limited to laser, microwave, and monopolar or bipolar electrosurgery may be used.

A sixth embodiment may provide for contouring of the perineum. As expressly disclosed in the method steps above, a portion of perineum skin may be removed sparing the underlying fascia and nerves. RF energy may then be applied to the fascia and other subcutaneous tissue. The shrinkage of the subcutaneous tissue and/or fascia will bring the epithelial edges closer together. The edges may be left “as is”, approximated with sutures or glue, or closed by any other manner known within the art. Such an embodiment is similar to that shown in FIGS. 2A-2C and analogous steps may be applied to the perineum. Although RF is the preferred energy source, any other types of energy known within the art including but not limited to laser, microwave, and monopolar or bipolar electrosurgery may be used.

A seventh embodiment may provide for contouring of the labia majora. As expressly disclosed in the method steps above, an incision may be made in the labia majora. The subcutaneous tissue may not be separated from the epithelium. RF energy may then be applied to the subcutaneous tissue. The shrinkage of the subcutaneous tissue and/or fascia shall contour the labia. Such an embodiment is similar to that shown in FIGS. 3A-3C and analogous steps may be applied to the labia majora. Although RF is the preferred energy source, any other types of energy known within the art including but not limited to laser, microwave, and monopolar or bipolar electrosurgery may be used.

As an alternative or addition, a portion of labial skin may be removed sparing the underlying fascia and nerves. RF energy may then be applied to the subcutaneous tissue and/or fascia. The shrinkage of the subcutaneous tissue and/or fascia will bring the labial skin edges closer together. The edges may be left “as is”, approximated with sutures or glue, or closed by any other manner known within the art. Such an embodiment is similar to that shown in FIGS. 2A-2C and analogous steps may be applied to the labial skin. Although RF is the preferred energy source, any other types of energy known within the art including but not limited to laser, microwave, and monopolar or bipolar electrosurgery may be used.

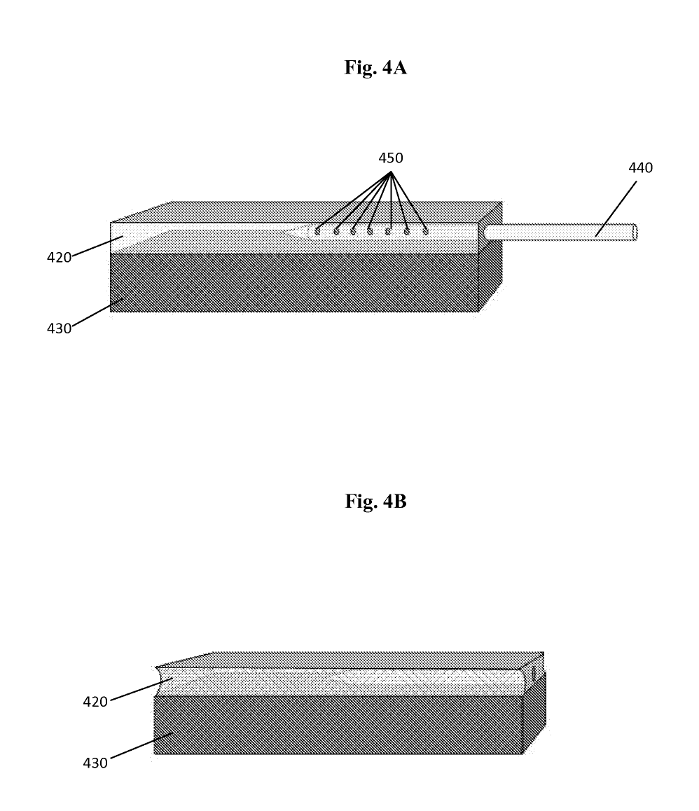

An eighth embodiment, depicted in FIG. 4A and FIG. 4B, may provide for transmucosal and transcutaneous contouring. As shown in FIG. 4A, pelvic tissues including but not limited to the vaginal mucosa, labia, prepuce, and/or perineum may be treated by the transcutaneous application of RF energy. In such an embodiment, RF energy may be applied to the tissue 430 (e.g. dermis, subcutaneous tissue, and/or fascia) below the mucosa or skin 420 without an incision being made or portions of the mucosa or skin 420 being removed. Application of such RF energy may preferably be via a needle, probe, or any other non-invasive instrument 440 known within the art. FIG. 4A depicts one embodiment performing the step of application of energy from one or more side ports 450 of a non-invasive means 440. As shown in FIG. 4B, the resultant shrinkage and changes to underlying tissue 430 shall shape the overlying structures as needed. Although RF is the preferred energy source, any other types of energy known within the art including but not limited to laser, microwave, and monopolar or bipolar electrosurgery may be used.

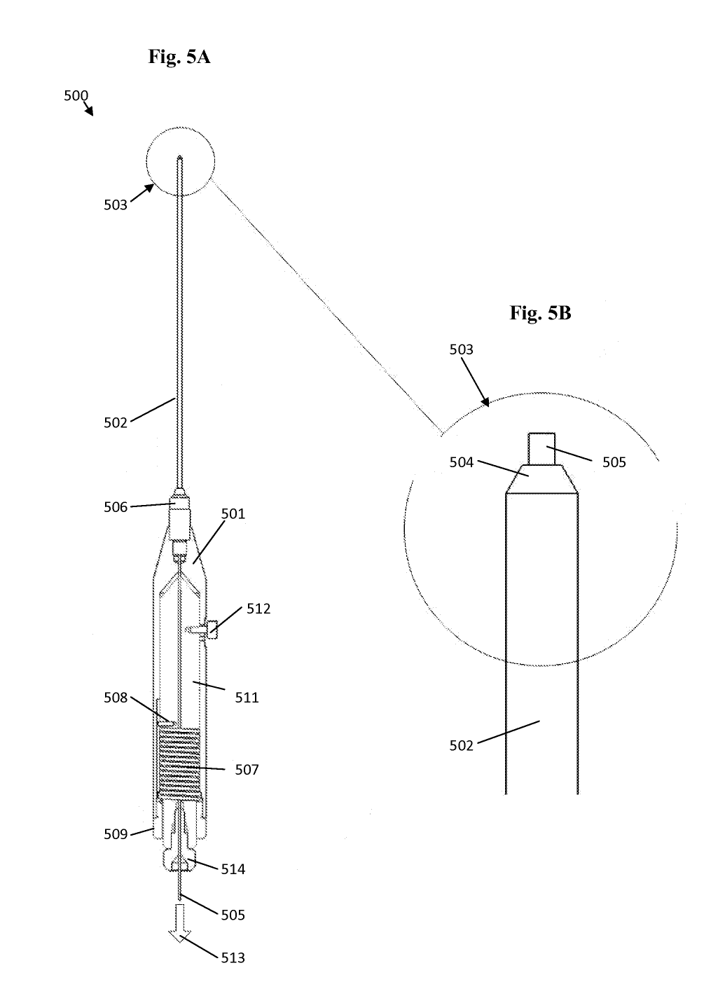

Another effective embodiment, similar to that shown in FIGS. 4A and 4B, may involve the use of a cannula and laser fiber. FIG. 5A depicts one potential and preferred embodiment of such a cannula and laser fiber device 500. In one embodiment, the device 500 may comprise an outer housing 501 secured to a cannula 502 having a distal portion 503. The distal portion 503 may comprise a distal tip 504 from which a laser fiber 505 may be extended and retracted. FIG. 5B illustrates a close-up view of the laser fiber 505 disposed in an extended state and protruding beyond the distal tip 504 of the cannula 502. The device 500 may comprise additional optional features to facilitate use, such optional features may include but are not limited to male/female Luer locks 506 for attaching the cannula to the outer housing 501, and a compression spring 507, clocking pin 508, spring cap 509, slide body 511, and slide limiter 512 providing for modes of extending and retracting the laser fiber 505. The slide limiter 512 may be in communication with the laser fiber 505 and be used to manually advance or motivate the laser fiber 505 between the retracted position and the extended position during a procedure. The slide limiter 512 may comprise a button, knob, finger rest surface, and the like that are well known for motivating movable components of surgical devices within the art. The laser fiber 505 may extend from the proximal end of the device 500 to a laser source 513 through a fiber locking screw 514.

Initially, the laser fiber 505 may be advanced to the distal portion 503 of the cannula 502 and thereafter the cannula 502 may be inserted through a small puncture or bodily cavity and then advanced to the desired treatment area. The cannula 502 may then be slightly retracted and/or the laser fiber 505 advanced disposing the distal end of the laser fiber 505 just beyond the distal tip 504 of the cannula 502. The laser fiber 505 may then be activated to deliver energy along the pathway of the cannula's 502 withdrawal. This delivery of energy may be supplied either continuously or in a pulsed manner. The energy being delivered through the distal end of the laser fiber 505 may be altered in power, pulse width, and/or rest time in order to provide differential treatment along the path of the device 500. Application of energy in this manner will result in a shaping or molding of the tissue rather than a uniform contraction. One example of use of such a device 500 and/or method may be in the vagina where application of a greater energy distally will help to create the normal taper of the vagina. In a preferred embodiment, energy may be applied in the form of 980 nm-1064 nm wavelength laser to be effective. However, other laser wavelengths and other forms of energy may replace the 980 nm laser. In a preferred embodiment, 810-1064 nm will be delivered at no less than 4 watts and no more than 25 watts. In the preferred embodiment pulse time will be no less than 0.1 second and no more than 2.5 seconds of continuous energy. However, in circumstances where the cannula 502 is kept in continuous motion (pulled out without stopping), the pulse may be equal to the length of time required to treat the entire cannula removal or insertion tract with the cannula 502 moving no slower than 0.25 cm per second. The preferred total energy delivered level to a single side of the vagina (anterior or posterior) is between 1000 joules and 4000 joules. In one variation of the preferred embodiment, the energy will be increased or decreased as the laser fiber 505 distal end approaches the opening of the vagina. If the vagina needs more tightening near the opening, the energy will be increased. If the apex of the vagina needs more shrinking than the opening of the vagina, the energy will be decreased as the laser fiber 505 distal end approaches the vaginal opening. The scope of the present invention includes the delivery of a constant power level, a continually or intermittently increasing power level, and/or a continually or intermittently decreasing power level during withdrawal of the cannula needle 502.

Preferably these power and/or pulse adjustments may be preset in the laser device 500. In one embodiment the laser power and/or pulse width will be serially increased or decreased each time the surgeon deactivates and then reactivates the laser (e.g. releases and steps back down on the laser pedal). Four typical presets start with the laser power at 12, 14, 17, and 19 watts and increase by 1 watt each time the surgeon reactivates the laser. The maximum increase is typically set between 5 and 10 watts. Once the maximum is reached, there may be no change in power with subsequent activations.

In another embodiment, the laser may be programmed to time out or deactivate once a predetermined maximum activation time, a predetermined maximum temperature, a predetermined maximum energy application level, and/or a predetermined maximum total energy delivered level has been reached. Such maximum times, temperatures, or energies may be preset in the laser device 500 or be predetermined by user input using mechanical or digital input devices or method that are abundantly common and well known in the art such as knobs, buttons, touch screens, digital displays, and the like. In an activation time-dependent embodiment, the laser device 500 may deactivate or time out after a maximum activation time is reached, wherein the maximum activation time may be in the range between 0.01 seconds and 5.00 seconds. In a preferred embodiment, the maximum activation time is 2.5 seconds. In a temperature-dependent embodiment, the laser device 500 may deactivate or time out after a maximum temperature is reached, wherein the maximum temperature near the tip of the cannula 502 may be in the range between 60 degrees Celsius and 99 degrees Celsius. In one energy-dependent embodiment, the laser device 500 may deactivate or time out after a predetermined maximum energy application level is reached. The preferred predetermined maximum energy application level is found within the range from 25 joules to 60 joules. In another energy-dependent embodiment, the laser device 500 may deactivate or time out after a predetermined maximum total energy delivered level is reached. The preferred predetermined maximum total energy delivered level is found within the range from 1000 joules to 4000 joules. After the laser device 500 has timed out or become deactivated after reaching the maximum time, temperature, energy application level, or total energy delivered the laser device 500 may be reactivated by the surgeon. In one embodiment, the surgeon or other user may reactivate the laser device 500 by merely releasing and then again pressing the activation switch or pedal controlling the laser power of the device 500. Additionally, while the disclosure describes a preferred method of energy application during withdrawal of the device 500, energy may also be applied or delivered during advancement of the device 500 as well. Although the present embodiment utilizes a laser as the preferred energy source, any other types of energy known within the art including but not limited to RF, microwave, and monopolar or bipolar electrosurgery may be used with such respective structures replacing the laser fiber 505.

In a preferred embodiment of the device 500, forward pressure or advancement of the cannula 502 may cause the laser fiber 505 to move back against a spring 507. Similarly, backward movement or withdrawal of the cannula 502 may cause the laser fiber 505 to be advanced or extended beyond the distal tip 504 of the cannula 502 by the biasing force of the spring 507. In an alternate embodiment of the device 500, the laser fiber 505 may require manual advancement against the biasing force of a spring 507 to advance or extend the distal end of the laser fiber 505 beyond the distal tip 504 of the cannula 502.

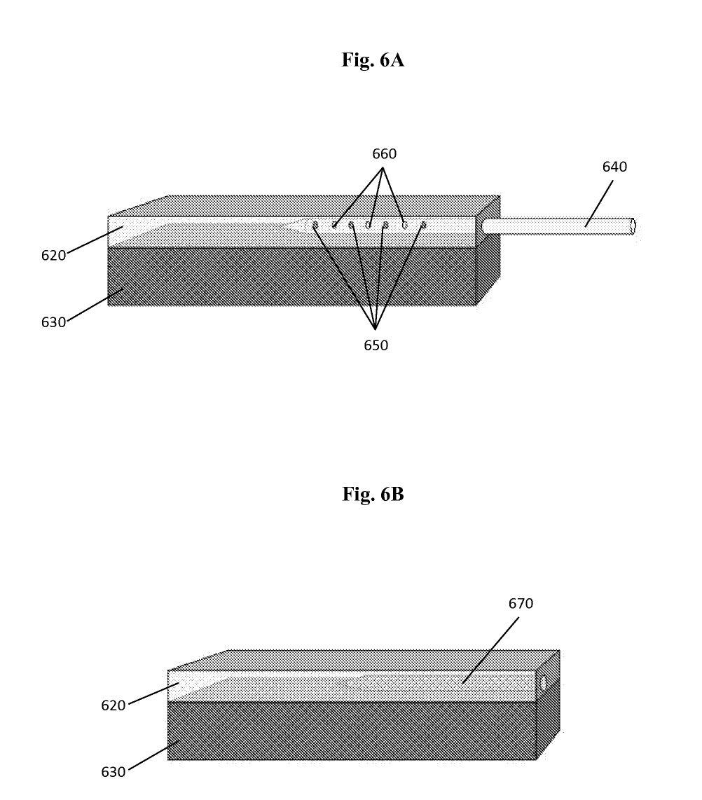

A ninth embodiment, depicted in FIG. 6A and FIG. 6B, may provide for applying minimally destructive energy directly or indirectly to the tissues 620 of the vagina and/or vulva, which may be followed by the implantation of stem cells 670. Such a “pretreatment” of energy may take the form of RF, microwave, laser, monopolar or bipolar electrosurgery, or any other surgical energy sources known within the art. As shown in FIG. 6A, the application of such energy may be delivered with or without an incision. Application of such RF energy may preferably be via a needle, probe, or any other non-invasive means 640 known within the art having application elements 650 such as ports, conduits, fibers, and the like respective to the specific type of energy source used. The pretreatment of energy creates an environment favorable to stem cells 670. Chemical pretreatment, via any known chemical agent(s), may also provide for minimal destruction and/or minimal injury. As shown in FIG. 6B, following pretreatment with an energy source the stem cells 670 may be implanted (i.e. treatment) through exit ports 660. Such implantation may be performed with a needle, via an incision, or any other means known within the art. The respective steps of pretreatment and treatment may be performed in either one stage or two separate stages and by one device or two separate devices.

A tenth embodiment may provide for a method of treating periurethral tissue. All method steps disclosed herein for decreasing the size or changing the shape of anatomical tissue, most particularly the ninth embodiment, may further be used in the treatment of periurethral tissue. Such treatments may improve the symptoms commonly associated with urinary incontinence.

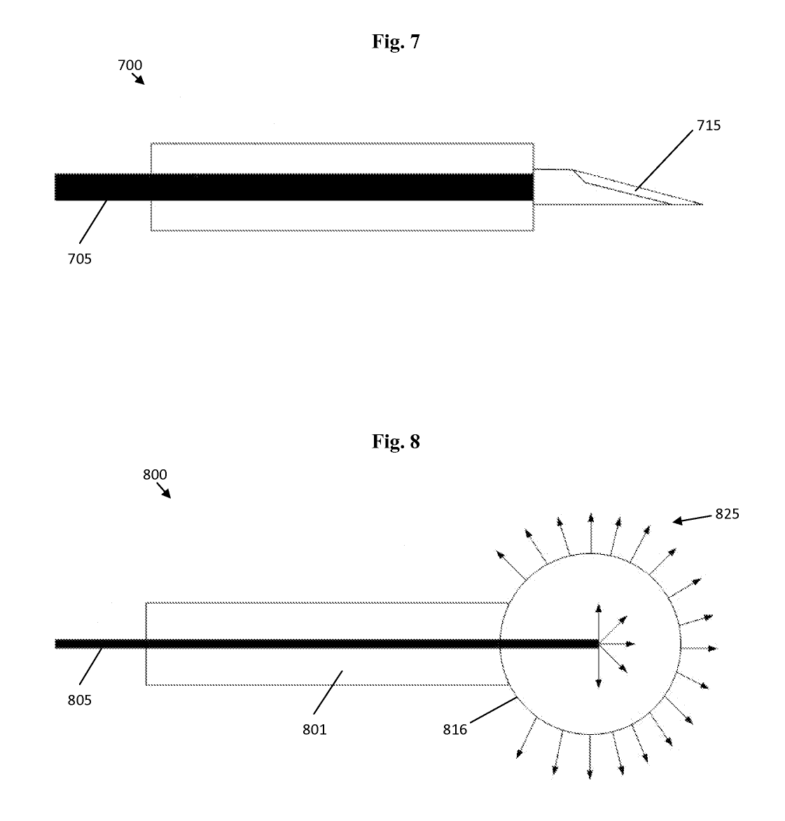

In an eleventh embodiment, the shaping or resizing of the vulva or other pelvic structure may be facilitated by the delivery of energy through a mechanical cutting instrument. One embodiment of such a device is depicted in FIG. 7 and may consist of a glass scalpel 700 or any other similar instrument known within the art. Such a glass scalpel 700 or equivalent device may be used to simultaneously create a mechanical cut or incision and deliver laser energy for coagulation and tissue treatment (e.g. shrinkage) purposes. In a preferred embodiment, CO₂ laser energy may be delivered by a laser fiber 705 to the scalpel cutting blade 715 in the range of 2 watts to 15 watts of continuous power. The energy shall be delivered to the blade as close to TEM₀₀ as possible.

In a twelfth embodiment as generally depicted in FIG. 8, low level laser energy may be delivered transmucosally to the vagina or other pelvic tissue. The use of such energy has been shown to increase cytochrome c oxidase production and reverse the effects of cellular inhibitors of respiration. Such steps may lead to healing of tissue, reduction of inflammation and pain, reduction in bladder problems such as urgency, frequency, and urinary incontinence, reshaping of tissue, and creation of a fertile environment for the potential implantation of stem cells. In one embodiment, the low level laser energy may be delivered via a device 800 comprising a laser fiber 805 disposed inside a vaginal probe 801. The probe 801 may be moved in and out of the vagina in order to deliver the energy to the appropriate tissues. The probe 801 may be made of glass, plastic, or any other material known within the art and may have a bulbous or “roller ball” type distal end 816. Such a “roller ball” structure may allow for the bulb to be illuminated 825 in a uniform 360 degree pattern or as close to such as possible. Multiple treatments may be necessary to achieve the desired effect. While 980 nm and 808 nm wavelength lasers are the preferred energy sources, other wavelengths, other energy sources including but not limited to RF, microwave, and monopolar or bipolar electrosurgery, and any combinations thereof may be used within the scope of the present invention.

In one method of use, the probe 801 may be inserted into the vagina until the distal end 816 reaches the vaginal apex. The laser fiber 805 may be in standby mode until the distal end 816 of the probe 801 is introduced into the vagina. Once the distal end 816 reaches the apex of the vagina, the laser fiber 805 may be put in ready mode. Once in ready mode, the laser 805 may be activated by stepping on a foot pedal. The user may step on the foot pedal once the distal tip 816 reaches the vaginal apex and then stay on the foot pedal while moving the probe 801 and distal end 816 in an “in and out” motion. In one embodiment, the probe 801 may be kept in constant motion for at least five minutes and reach a total output of 3200 J. The user may then release the foot pedal prior to the removal of the probe 801 and distal end 816 to place the laser 805 back in standby mode prior to extraction of the device 800.

While the above description contains much specificity, these should not be construed as limitations on the scope of any embodiment, but as exemplifications of the presently preferred embodiments thereof. Many other ramifications and variations are possible within the teachings of the various embodiments.

Thus the scope of the invention should be determined by the appended claims and their legal equivalents, and not by the specific examples given.

What is claimed is:

1. A method for reshaping tissue, said method comprising the steps of:
providing a device comprising a cannula needle and a laser fiber coaxially disposed within said cannula needle, wherein said laser fiber may be disposed in a retracted position with a distal end of said laser fiber disposed within a distal tip of said cannula needle and an extended position wherein said distal end of said laser fiber protrudes beyond said distal tip of said cannula needle;
advancing said cannula needle into said tissue wherein said laser fiber is in said retracted position, wherein said cannula needle forms a pathway within said tissue during said advancement;
motivating said laser fiber into said extended position;
activating said laser fiber thereby delivering laser energy to said adjacent tissue; and
withdrawing said cannula needle along said pathway providing for delivery of said laser energy along said pathway during said withdrawal.

2. The method of claim 1, further comprising the step of:
deactivating said laser fiber and thereby terminating delivery of said laser energy before said distal tip of said laser fiber is withdrawn from said tissue.

3. The method of claim 2, further comprising the step of:
reactivating and thereafter deactivating said laser fiber one or more times before said distal tip of said laser fiber is withdrawn from said tissue.

4. The method of claim 1, wherein said step of activating said laser fiber delivers said laser energy at a constant power level during said withdrawal.

5. The method of claim 1, wherein said step of activating said laser fiber delivers said laser energy at a continually increasing power level during said withdrawal.

6. The method of claim 1, wherein said step of activating said laser fiber delivers said laser energy at a continually decreasing power level during said withdrawal.

7. The method of claim 1, wherein said step of activating said laser fiber delivers said laser energy in a pulsed manner, wherein said pulsed manner comprises energy pulses of a duration in the range between 0.1 seconds and 2.0 seconds.

8. The method of claim 1, further comprising the step of:
deactivating said laser fiber and thereby terminating delivery of said laser energy after a predetermined maximum activation time, wherein said predetermined maximum activation time is in the range between 0.01 seconds and 5.00 seconds.

9. The method of claim 1, further comprising the step of:
deactivating said laser fiber and thereby terminating delivery of said laser energy after said tissue reaches a predetermined maximum temperature, wherein said predetermined maximum temperature is in the range between 60 degrees Celsius and 90 degrees Celsius.

10. The method of claim 1, further comprising the step of:
deactivating said laser fiber and thereby terminating delivery of said laser energy after a predetermined maximum energy application level of said laser energy is reached, wherein said predetermined maximum energy application level is in the range from 25 joules to 60 joules.

11. The method of claim 1, further comprising the step of:
deactivating said laser fiber and thereby terminating delivery of said laser energy after a predetermined maximum total energy delivered level of said laser energy is reached, wherein said predetermined maximum total energy delivered level is in the range from 1000 joules to 4000 joules.

12. The method of claim 1, wherein said method is performed in a pelvic region of a patient.

13. The method of claim 12, wherein said pelvic region of said patient comprises the vagina.

14. A method for reshaping pelvic tissue, said method comprising the steps of:
providing a device comprising a cannula needle and a laser fiber coaxially disposed within said cannula needle, wherein said laser fiber may be disposed in a retracted position with a distal end of said laser fiber disposed within a distal tip of said cannula needle and an extended position wherein said distal end of said laser fiber protrudes beyond said distal tip of said cannula needle;
advancing said cannula needle into said pelvic tissue wherein said laser fiber is in said retracted position, wherein said cannula needle forms a pathway within said pelvic tissue during said advancement;
motivating said laser fiber into said extended position;
activating said laser fiber thereby delivering laser energy to said pelvic tissue; and
withdrawing said cannula needle along said pathway providing for delivery of said laser energy along said pathway during said withdrawal;
wherein said laser fiber is biased by a spring into said extended position, wherein during said advancement of said cannula needle said laser fiber is moved back against said spring into said retracted position and during said withdrawal of said cannula needle said spring biases said laser fiber into said extended position.

15. The method of claim 14, further comprising the step of:
deactivating said laser fiber and thereby terminating delivery of said laser energy after a predetermined maximum activation time, wherein said predetermined maximum activation time is in the range between 0.01 seconds and 5.00 seconds.

16. The method of claim 14, further comprising the step of:
deactivating said laser fiber and thereby terminating delivery of said laser energy after said pelvic tissue reaches a predetermined maximum temperature, wherein said predetermined maximum temperature is in the range between 60 degrees Celsius and 90 degrees Celsius.

17. The method of claim 14, further comprising the step of:
deactivating said laser fiber and thereby terminating delivery of said laser energy after a predetermined maximum energy application level of said laser energy is reached, wherein said predetermined maximum energy application level is in the range from 25 joules to 60 joules.

18. The method of claim 14, further comprising the step of:
deactivating said laser fiber and thereby terminating delivery of said laser energy after a predetermined maximum total energy delivered level of said laser energy is reached, wherein said predetermined maximum total energy delivered level is in the range from 1000 joules to 4000 joules.

19. A method for reshaping pelvic tissue, said method comprising the steps of:
providing a device comprising a cannula needle and a laser fiber coaxially disposed within said cannula needle, wherein said laser fiber may be disposed in a retracted position with a distal end of said laser fiber disposed within a distal tip of said cannula needle and an extended position wherein said distal end of said laser fiber protrudes beyond said distal tip of said cannula needle;
advancing said cannula needle into said pelvic tissue wherein said laser fiber is in said retracted position, wherein said cannula needle forms a pathway within said pelvic tissue during said advancement;
motivating said laser fiber into said extended position;
activating said laser fiber thereby delivering laser energy to said pelvic tissue; and
withdrawing said cannula needle along said pathway providing for delivery of said laser energy along said pathway during said withdrawal;
wherein said laser fiber is manually motivated between said retracted position and said extended position by a slide limiter on said device.

20. The method of claim 19, further comprising the step of:
deactivating said laser fiber and thereby terminating delivery of said laser energy after a predetermined maximum activation time, wherein said predetermined maximum activation time is in the range between 0.01 seconds and 5.00 seconds.

21. The method of claim 19, further comprising the step of:
deactivating said laser fiber and thereby terminating delivery of said laser energy after said pelvic tissue reaches a predetermined maximum temperature, wherein said predetermined maximum temperature is in the range between 60 degrees Celsius and 90 degrees Celsius.

22. The method of claim 19, further comprising the step of:
deactivating said laser fiber and thereby terminating delivery of said laser energy after a predetermined maximum energy application level of said laser energy is reached, wherein said predetermined maximum energy application level is in the range from 25 joules to 60 joules.

23. The method of claim 19, further comprising the step of:
deactivating said laser fiber and thereby terminating delivery of said laser energy after a predetermined maximum total energy delivered level of said laser energy is reached, wherein said predetermined maximum total energy delivered level is in the range from 1000 joules to 4000 joules.
